(19) United States
(12) Patent Application Publication
(10) Pub. No.: US 2003/0124084 A1
(43) Pub. Date: Jul. 3, 2003

(54) Use of dehydroepiandrosterone and/or precursors or derivatives thereof to improve papery aspect of the skin

(76) Inventors: Olivier De Lacharriere, Paris (FR); Stephanie Nouveau, Boulogne (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/169,101

(22) PCT Filed: Jan. 8, 2001
(86) PCT No.: PCT/FR01/00043

(30) Foreign Application Priority Data
Jan. 12, 2000 (FR) 0000349

(51) Int. Cl. A61K 7/06; A61K 35/78; A61K 31/56

(52) U.S. Cl. 424/74; 424/757; 424/773; 514/178

(57) ABSTRACT

The present invention relates to the use of DHEA and/or of at least one precursor or derivative thereof in or for the manufacture of a composition for topical application to the skin, as an agent for preventing or reducing the weathered appearance of the skin.

The present invention relates to the use of DHEA and/or of at least one precursor or derivative thereof in or for the manufacture of a composition for topical application to the skin, as an agent for preventing or reducing the weathered appearance of the skin, in particular in the treatment of ageing of the skin.

Ageing of the skin results from the effects of intrinsic and extrinsic factors on the skin. The changes in the skin resulting from intrinsic or physiological ageing are the consequence of a genetically programmed senescence involving endogenous factors. This intrinsic ageing gives rise especially to a slowing-down of renewal of the skin cells. Histologically, the skin is thinner overall, both at the epidermal and dermal levels. The density of the fibrous macromolecules in the dermis (elastine and collagen) is reduced. In contrast, extrinsic ageing entails histopathological changes such as an excessive accumulation of elastic material in the upper dermis and degeneration of the collagen fibers.

Clinically, the signs of ageing are generally reflected by the appearance of wrinkles and fine lines, a slackening of the cutaneous and subcutaneous tissues, a loss of skin elasticity and atonia of the skin texture. The loss of firmness and tonicity of the skin, like wrinkles and fine lines, is at least partly explained by dermal and epidermal atrophy and also by a flattening of the dermoepidermal formation; the skin is thinner and more flaccid, and the epidermis reduces in thickness.

In addition, the complexion of the skin is generally modified; it appears paler and yellower; this appears to be due essentially to a disorganization of the microcirculation (less hemoglobin in the papillary dermis). Another clinical sign of ageing is the dry and coarse appearance of the skin, which is due essentially to a more pronounced level of desquamation; by scattering light rays, these squamae also contribute toward a somewhat gray appearance of the complexion.

Furthermore, many colored and/or darker marks appear at the surface of the skin, and more especially on the hands, giving the skin a nonuniform appearance. In general, these marks are due to a large production of melanin in the epidermis and/or dermis of the skin. In certain cases, these marks may become cancerous. Moreover, diffuse irritations and sometimes telangiectasia occur in certain areas of the skin.

Some of these signs are more particularly associated with intrinsic or physiological ageing, i.e. age-related ageing, whereas others are more specific to extrinsic ageing, i.e. ageing generally brought about by the environment; this more particularly involves photoageing due to exposure to sunlight, light or any other radiation.

Another sign of ageing of the skin that constitutes a major criterion in the degradation of the appearance of the skin is the accentuation of the weathered appearance.

The weathered appearance is characterized by a change in the visual appearance and also the touch behavior of the skin. More specifically, the skin visually takes on the appearance of cigarette paper, giving it an appearance similar to that of a sheet of papyrus. In addition, when it is gently pinched between the thumb and index finger, the skin forms numerous thin, sharp folds having the appearance of creased paper. Finally, the feel of the skin shows that its superficial portions appear to be floating on the deeper portions, giving the skin, at the very advanced stage of a weathered appearance, the look of crumpled paper.

The cosmetic or dermatological active agents that are recognized as being effective on some of the signs of ageing of the skin do not necessarily have an effect on the weathered appearance of the skin. On the contrary, the only active agent known to date to be capable of limiting this increase in the weathered appearance is retinoic acid.

However, the topical use of retinoic acid is not without side effects. At low concentration, it thus frequently causes stinging, or even itching or erythema, giving rise to an unacceptable level of discomfort for cosmetic use and occasionally leading patients to interrupt their treatment in the case of a therapeutic use.

Thus, one aim of the present invention is to propose a novel active agent for improving the weathered appearance of the skin without having the side effects of retinoic acid, and in particular without causing skin irritation.

The Applicant has found, unexpectedly, that DHEA and/or at least one precursor or derivative thereof allow this aim to be achieved.

DHEA, or dehydroepiandrosterone, is a natural steroid produced essentially by the adrenal glands. It is known for its anti-ageing properties, associated with its ability to promote epidermal keratinization (JP-07 196 467) and to combat osteoporosis (U.S. Pat. No. 5,824,671), or alternatively in the treatment of dry skin, on account of its ability to increase the endogenous production and secretion of sebum and to strengthen the skin's barrier effect (U.S. Pat. No. 4,496,556). DHEA is also described in the treatment of obesity and diabetes (WO 97/13500). It has also been proposed to use DHEA sulfate against alopecia (JP-60 142 908) and to treat various signs of ageing such as wrinkles, loss of radiance of the skin and slackening of the skin (EP-0 723 775).

However, it has never before been suggested that DHEA and/or at least one precursor or derivative thereof could have an effect on the weathered appearance of the skin.

One subject of the present invention is thus the use of DHEA and/or of at least one precursor or derivative thereof in a cosmetic composition for topical application to the skin, to prevent or reduce the weathered appearance of the skin.

A subject of the invention is also the use of DHEA and/or of at least one precursor or derivative thereof for the manufacture of a composition for topical application to the skin, intended to prevent or reduce the weathered appearance of the skin.

The DHEA that may be used according to the invention is available, for example, from the companies Sigma and Akzo Nobel.

The expression “DHEA precursors” means its immediate biological precursors or substrates, and also its chemical precursors. Examples of biological precursors are cholesterol, pregnenolone, 17α-hydroxy pregnenolone, 5-androstenediol, DHEA sulfate, 17α-hydroxy pregnenolone sulfate and 5-androstenediol sulfate, this list not intending to be limiting. Examples of chemical precursors are sapogenins such as diosgenin (or spirost-5-ene-3-β-ol), hecogenin, smilagenin and sarsapogenin, and also natural extracts containing them, in particular fenugrec and extracts of Dioscorea plants such as wild yam root, this list not intended to be limiting.

The expression “DHEA derivatives” means both its metabolic derivatives and its chemical derivatives. Metabolic derivatives that may be mentioned especially include 5-androstene-3β-17β-diol (or adiol), 5-androstene-3β-17β-diol sulfate and 4-androstene-3,17-dione, this list not intended to be limiting. Chemical derivatives that may be mentioned especially include salts, in particular water-soluble salts, such as DHEA sulfate. Mention may also be made of esters, such as the hydroxycarboxylic acid esters of DHEA described in U.S. Pat. No. 5,736,537 or other esters such as DHEA salicylate, acetate, valerate or enanthate.

The composition containing DHEA and/or at least one precursor or derivative thereof is suitable for topical use and it thus contains a physiologically acceptable medium, i.e. a medium that is compatible with the skin.

This composition may contain from 10-6 to 10% by weight, advantageously from 0.1% to 5% by weight and better still about 1% by weight of DHEA and/or of the precursor or derivative thereof, relative to the total weight of the composition.

It may be in any presentation form normally used for topical application, especially in the form of an aqueous or aqueous-alcoholic solution, an oil-in-water or water-in-oil or multiple emulsion, an aqueous gel or a liquid, pasty or solid anhydrous product.

This composition may be more or less fluid and may have the appearance of a white or colored cream, an ointment, a milk, a lotion, a serum, a paste or a mousse. It may optionally be applied to the skin in the form of an aerosol. It may also be in solid form, and for example in the form of a stick. It may be used as a care product and/or as a makeup product.

In a known manner, the composition according to the invention may also contain adjuvants that are common in cosmetics and dermatology, such as hydrophilic or lipophilic gelling agents, hydrophilic or lipophilic active agents, preserving agents, antioxidants, solvents, fragrances, fillers, screening agents, pigments, odor absorbers and dye stuffs. The amounts of these various adjuvants are those conventionally used in the fields under consideration, and for example from 0.01% to 20% of the total weight of the composition. Depending on their nature, these adjuvants may be introduced into the fatty phase, into the aqueous phase, into lipid vesicules and/or into nanoparticles.

Needless to say, a person skilled in the art will take care to select these optional additional active or nonactive compounds, and/or the amount thereof, such that the advantageous properties of the DHEA and/or of the precursors or derivatives thereof, are not, or are not substantially, adversely affected by the envisaged addition.

When the composition according to the invention is an emulsion, the proportion of the fatty phase can range from 0.5% to 80% by weight and preferably from 5% to 50% by weight relative to the total weight of the composition. The oils, emulsifiers and coemulsifiers used in the composition in emulsion form are chosen from those conventionally used in the field under consideration. The emulsifier and coemulsifier are present in the composition in a proportion ranging from 0.3 to 30% by weight and preferably from 0.5 to 20% by weight relative to the total weight of the composition.

As oils that may be used in the invention, mention may be made of mineral oils (liquid petroleum jelly), oils of plant origin (avocado oil, soybean oil), oils of animal origin (lanolin), synthetic oils (perhydrosqualene), silicone oils (cyclomethicone) and fluoro oils (perfluoropolyethers). Fatty alcohols (cetyl alcohol), fatty acids and waxes (carnauba wax, ozokerite) may also be used as fatty substances.

As emulsifiers and coemulsifiers that may be used in the invention, examples that may be mentioned include fatty acid esters of polyethylene glycol such as PEG-20 stearate, and fatty acid esters of glyceryl such as glyceryl stearate.

Hydrophilic gelling agents that may be mentioned in particular include carboxyvinyl polymers (carbomer), acrylic copolymers such as acrylate/alkylacrylate copolymers, polyacrylamides, polysaccharides, natural gums and clays, and lipophilic gelling agents that may be mentioned include modified clays, for instance bentones, metal salts of fatty acids, hydrophobic silica and polyethylenes.

Active agents that may be used especially include keratolytic and/or desquamanting agents, depigmenting agents, UV screening agents, free-radical scavengers and mixtures thereof. In the event of incompatibility, at least some of the active agents may be incorporated into spherules, especially ionic or nonionic vesicules and/or nanoparticles (nanocapsules and/or nanospheres), such that the mutually incompatible active agents are isolated from each other in the composition.

These compositions especially constitute protective, treatment or care creams for the face, for the hands or for the body, protective or care body milks, lotions, gels or mousses for skin care or skin treatment, bath compositions, foundations and tinted creams. In these latter cases, the composition contains pigments.

The invention will now be illustrated with the aid of the nonlimiting examples that follow.

### EXAMPLE 1

### DHEA-Based Composition

The composition below was prepared, in which the proportions of the various constituents are indicated in percentages by weight:

| | | |
|---|---|---|
| DHEA | | 2% |
| Propylene glycol isostearate | | 13% |
| Polyethylene glycol (8 EO) | | 5% |
| Propylene glycol | | 3% |
| Pentylene glycol | | 3% |
| Glyceryl stearate and polyethylene glycol | | 5% |
| stearate (100 EO) | | |
| Oxyethylenated sorbitan monostearate (20 EO) | | 0.5% |
| Oxyethylenated (20 EO) oxypropylenated (5 PO) | | 1% |
| cetyl alcohol | | |
| Gelling agents | | 0.5% |
| C₁₂₋₁₅ alkyl benzoates | | 4% |
| Ethanol | | 3% |
| Sodium hydroxide | | 0.12% |
| Preserving agents | | 0.7% |
| Water | qs | 100% |

### EXAMPLE 2

### Demonstration of the Effect of DHEA on the Weathered Appearance

a—Protocol

A gelled oil-in-water emulsion containing 1% by weight of DHEA as sole active agent was tested in comparison with a vehicle consisting of an identical emulsion but not containing DHEA.

The products were tested on a group of forty women aged from 55 to 70. Half of the individuals applied the vehicle and the other half applied the DHEA-based emulsion, the choice of the individuals being made according to a computer-generated randomization. The emulsion was applied in the morning and in the evening for four months to the back of only one hand, the other hand serving as a control. The amounts applied were controlled by weighing the tubes at the start and end of the test.

The weathered appearance is assessed clinically under blind conditions on the back of each hand, at the start of the test and after four months, by the same clinician. The weathered appearance is represented by a clinical score ranging from 0 (weathered appearance absent or relatively unpronounced) to 9 (very pronounced weathered appearance).

The statistical analysis is performed by Wilcoxon tests.

b—Results

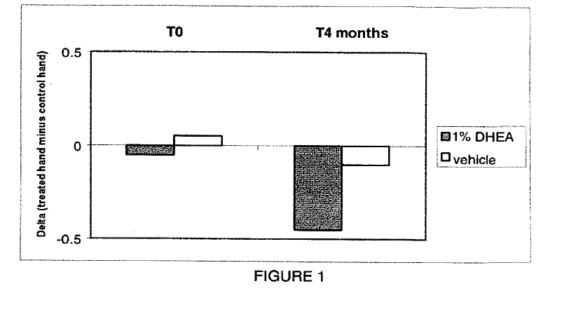

The results are illustrated by the attached FIG. 1.

This figure shows that, at the initial time (To), the differences in the scores of the weathered appearance between the two hands are virtually nonexistent, irrespective of the group of individuals under consideration.

After treatment for four months, this difference remains virtually nonexistent, or in any case nonsignificant, for the group treated with the vehicle (placebo). In contrast, for the group treated with DHEA, a difference in scores is observed between the two hands: the score for the weathered appearance is lower on the hand treated with DHEA than on the control hand. This decrease is significant at p=0.05.

It may be deduced therefrom that the topical application of DHEA improves the appearance of the skin by reducing the score for the weathered appearance.

### EXAMPLE 2

### Effect of DHEA on the Elasticity, Texture and Flaccidness of the Skin

The aim of this example is to demonstrate that the improvement in the weathered appearance of the skin is not associated with a possible effect of DHEA on the elasticity, texture and/or flaccidness of the skin.

a—Protocol

This test was performed simultaneously with the one described in example 2, by applying the same products, onto the same groups of individuals, and under the same conditions, except that the products were applied to the skin of the face.

The texture of the skin and the flaccidness were assessed under blind conditions by the same clinician and are represented by clinical scores ranging from 0 (unpronounced or relatively unpronounced parameter) to 9 (very pronounced parameter).

The elasticity of the skin was assessed, at the start of the test and after four months, using the apparatus available under the tradename Dermal Torque Meter® from DTM® Dia-Stron Limited, Andover, Hampshire, United Kingdom. This apparatus makes it possible to study the response of the skin to a deformation applied in the direction parallel to its surface, as described in Escoffier C et al., Age-Related Mechanical Properties of Human Skin: an in Vivo Study, J. Invest. Dermatol., 1989, 93:353-357.

The statistical analysis was performed by Wilcoxon tests.

b—Results

The results are given in table 1 below.

TABLE 1

| | |
|---|---|
| DHEA | PLAEBO |

| PARAMETER | T0 | T4 | T0 | T4 | SIGNIFICANCE |
|---|---|---|---|---|---|
| Texture | 4 | 3.8 | 4.05 | 3.4 | P = 0.8 |
| Flaccidness | 4.45 | 4.15 | 4.45 | 4.05 | P = 0.72 |
| Elasticity | 0.77 | 0.66 | 0.69 | 0.65 | P = 0.77 |

It emerges from table 1 above that the topical application of DHEA does not significantly modify the texture, elasticity or flaccidness of the skin. The improvement in the weathered appearance observed in example 2 is thus independent of the variation of these parameters.

1. The use of DHEA and/or of at least one precursor or derivative thereof in a cosmetic composition for topical application to the skin, to prevent or reduce the weathered appearance of the skin.

2. The use of DHEA and/or of at least one precursor or derivative thereof for the manufacture of a composition for topical application to the skin, intended to prevent or reduce the weathered appearance of the skin.

3. The use as claimed in claim 1 or 2, characterized in that said precursor is a metabolic precursor chosen from: cholesterol, pregnenolone, 17α-hydroxy pregnenolone, 5-androstenediol, DHEA sulfate, 17α-hydroxy pregnenolone sulfate and 5-androstenediol sulfate.

4. The use as claimed in claim 1 or 2, characterized in that said precursor is a chemical precursor chosen from sapogenins and natural extracts containing them.

5. The use as claimed in claim 4, characterized in that said chemical precursor is chosen from diosgenin, hecogenin, smilagenin and sarsapogenin.

6. The use as claimed in claim 4, characterized in that said natural extract is chosen from fenugrec and extracts of Dioscorea plants.

7. The use as claimed in claim 6, characterized in that said extract of Dioscorea plants is an extract of wild yam root.

8. The use as claimed in claim 1 or 2, characterized in that said derivative is a metabolic derivative chosen from: 5-androstene-3β-17β-diol (or adiol), 5-androstene-3β-17β-diol sulfate and 4-androstene-3,17-dione.

9. The use as claimed in claim 1 or 2, characterized in that said derivative is a chemical derivative chosen from DHEA salts and esters.

10. The use as claimed in any one of the preceding claims, characterized in that the DHEA and/or the precursor or derivative thereof are present in an amount ranging from 10⁻⁶ to 10% by weight relative to the total weight of the composition.

11. The use as claimed in claim 10, characterized in that the DHEA and/or the precursor or derivative thereof are present in an amount ranging from 0.1% to 5% by weight relative to the total weight of the composition.

12. The use as claimed in claim 11, characterized in that the DHEA and/or the precursor or metabolic derivative thereof are present in an amount of about 1% by weight relative to the total weight of the composition.
